(19) United States
(12) Patent Application Publication
(10) Pub. No.: US 2016/0030365 A1
(43) Pub. Date: Feb. 4, 2016

(54) PHARMACEUTICAL OR COSMETIC COMPOSITION SUITABLE TO PRESERVE EPITHELIAL STEM CELLS

(71) Applicant: GIULIANI S.P.A., Milano (IT)

(72) Inventors: Giammaria Giuliani, Milano (IT); Anna Benedusi, Milano (IT); Barbara Marzani, Carbonara Al Ticino (IT); Sergio Baroni, Villa D'adda (IT); Ralf Paus, Hamburg (DE); Yuval Ramot, Mevasseret-Zion (IL)

(73) Assignee: Giuliani S.p.A., Milano (IT)

(21) Appl. No.: 14/783,157

(22) PCT Filed: Apr. 9, 2014
(86) PCT No.: PCT/EP14/57166

§ 371 Date: Oct. 8, 2015

(30) Foreign Application Priority Data
Apr. 9, 2013 (IT) MI2013A000554

(51) Int. Cl. A61K 31/132; A61Q 7/00; A61K 35/36; A61Q 19/00; A61K 8/41; A61K 9/00

(52) U.S. Cl. CPC A61K 31/132; A61K 8/41; A61K 9/0014; A61K 35/36; A61Q 19/00; A61Q 7/00

(57) ABSTRACT

The invention concerns the use of compounds of formula (I) R-N¹-spermidine, or 1,4-butandiamine,N-(3-amino propyl)-N¹—R, (I) H₂N—(CH₂)₃—N¹ (R)—(CH₂)₄—NH₂, as such or in the form of pharmaceutically acceptable derivatives, as the active principle in a pharmaceutical or cosmetic composition to preserve and protect epithelial stem cells, and progenitor cells that derive from them, through topical application on the skin or through the use of compounds in cell cultures. Hence, the invention especially concerns the use of compounds of formula (I) to treat cicatricial alopecia, and to stimulate and accelerate tissue repair and healing of both wounds and epidermal scars.

### FIELD OF THE INVENTION

This invention concerns a pharmaceutical or cosmetic composition suitable to preserve epithelial stem cells in mammals, particularly in humans.

### BACKGROUND OF THE INVENTION

The epidermis is constantly renewed to preserve its homeostasis and tissue functions. The above-mentioned renewal process causes proliferation of epithelial stem cells. During the physiological aging processes, there is a progressive loss of regular skin homeostasis and certain essential tissue processes, such as epidermal regeneration, cicatrisation and healing capacity, naturally slow down. To maintain skin function, stem cells can play an essential role in preventing premature skin aging. However, during the aging process stem cells undergo age-related changes and progressive loss of their function.

The skin and hair follicles have a structure that is organised by molecular mechanisms that regulate renewal, proliferation and migration of stem cells. Each hair follicle is made up of a permanent portion that includes sebaceous glands and the underlying bulge area where the hair bulb is situated, and a portion that undergoes renewal cycles, such as anagen (active growth phase), catagen (remodelling phase) and, finally, telogen (quiescent phase). Two important elements that control the hair follicle cycle are follicular epithelial stem cells situated in the bulge area of the hair follicle, and specialised mesenchymal cells that constitute the follicular papilla.

Epithelial stem cells are multipotent. They produce daughter cells that either migrate inwards to turn into progenitor cells of the hair matrix, originating the hair shaft, or migrate outwards to turn into epidermal progenitor cells during cicatrisation of wounds.

There are various reasons that can cause hair growth to either slow down or stop. The production of hair fibre can stop, for instance, because matrix cells have depleted their proliferating capacity. This leads to assume that the proliferative capacity of matrix cells is finally defined at the start of a new hair cycle, and that new matrix cells cannot be generated during the entire growth phase.

Stem cells can continuously generate new matrix cells. The production of hair fibre can end when the stem cells are instructed to stop generation of a new progeny. Certain types of alopecia, classified as cicatricial or scratch alopecia, such as lichen planopilaris, frontal fibrosing alopecia, chronic cutaneous lupus erythematosus, keratosis follicularis, spinulosa decalvans or folliculitis decalvans, are disorders that cause hair follicle stem cell destruction in the bulge area and permanent loss of hair.

Hence, preventing the destruction of hair follicle stem cells can allow regrowth of hair in patients with disorders associated with the destruction of stem cells. The preservation of epithelial stem cells protects hair follicle progenitor cells, allowing the hair follicle to maintain its regenerating capacity with a subsequent regrowth of hair.

A main purpose of this invention is to provide means that can protect hair follicle progenitor cells by preserving epithelial stem cells in order to preserve the hair follicle and to enable it to regenerate, firstly to treat cicatricial alopecia.

A further purpose of this invention is to provide means to protect epithelial stem cells capable to become progenitors of epidermal cells, so as to promote epidermal regeneration processes.

In particular, according to said purposes of this invention reference is made to an action that stimulates and accelerates tissue repair in wound healing such as, for example, cuts, burns, scars, subcutaneous injury wounds and surgical wounds. WO2005013932 of the same Applicant describes a composition for pharmaceutical, dietetic or cosmetic use suitable to slow down aging of skin and of skin annexes by including spermine, spermidine or their salts as active ingredient. Said document especially describes experimental clinical studies related to the effects of said polyamines on elasticity, hydration and the cell renewal action of skin. However, this paper neither describes nor suggests any effects of the active compounds spermine, spermidine or their salts on either stem cells or progenitor cells.

It must also be mentioned that in the case of formulations for topical use according to WO2005013932, spermidine, like other polyamines, is subjected to oxidation because, during topical application on the skin, it remains in extensive contact with the air for a certain period of time before being absorbed by the skin to perform its action. The possible oxidation of spermidine during said period prior to absorption would produce oxidation products that are not active anymore.

### SUMMARY OF THE INVENTION

According to this invention, it has now been surprisingly found that it is possible to produce technical effects based on the above stated purposes of this invention, basically preservation and protection of epithelial stem cells and of progenitor cells that descend from them, using a compound of general formula (I): R—N¹-spermidine as defined below, either as such or as a pharmaceutically acceptable derivative, such as a salt, through topical application on the skin of a suitable composition that contains it.

The subject of the invention is the use of compounds of formula (I) R—N¹-spermidine, or 1,4-butandiamine, N-(3-amino propyl)-N¹—R,

H₂N—(CH₂)₃—N¹(R)—(CH₂)₄—NH₂ (I)

wherein R is a substituent bound to the secondary amine function of spermidine, selected from:

saturated or unsaturated, linear or branched alkyl groups constituted by 1 to 6 carbon atoms, wherein one or more carbon atoms are optionally substituted by fluorine, such as methyl, ethyl, trifluoromethyl, trifluoroethyl, propyl, isopropyl, butyl, isobutyl, pentyl, hexyl, ethylene, vinyl, propylene, butylene;

aryl or arylalkyl groups, such as phenyl, naphthyl, benzyl, tolyl, wherein one or more carbon atoms are optionally substituted by fluorine, and wherein said arylalkyl groups comprise saturated or unsaturated, linear or branched alkyl groups constituted by 1 to 6 carbon atoms, wherein one or more carbon atoms are optionally substituted by fluorine, namely methyl, ethyl, trifluoromethyl, trifluoroethyl, propyl, isopropyl, butyl, isobutyl, pentyl, hexyl, ethylene, vinyl, propylene, butylene;

saturated or unsaturated cycloalkyl groups constituted by 3 to 8 carbon atoms, optionally substituted by saturated or unsaturated, linear or branched alkyl groups constituted by 1 to 6 carbon atoms, wherein one or more carbon atoms are optionally substituted by fluorine, such as methyl, ethyl, trifluoromethyl, trifluoroethyl, propyl, isopropyl, butyl, isobutyl, pentyl, hexyl, ethylene, vinyl, propylene, butylene; or a corresponding pharmaceutically acceptable salt, said use being directed to the preservation and protection of epithelial stem cells and progenitor cells originating therefrom.

The epithelial stem cells in question belong to mammals in general, particularly to humans.

To this end, the invention proposes a first particular use based on topical application of compounds of formula (I) on the scalp to preserve and protect the hair follicle progenitor cells that have originated from epithelial stem cells in order to preserve the hair follicle and enable it to regenerate, to treat cases of cicatricial alopecia.

From said effect, the invention also proposes an additional particular use through topical application of compounds of formula (I) directly on the skin to preserve and protect epithelial stem cells that can become progenitors of epidermal cells in order to encourage epidermal regeneration processes. Consistently with said purpose of this invention, we particularly refer to the use of compounds of formula (I) to stimulate and accelerate tissue repair and healing of both wounds and epidermal scars.

Moreover, the compounds of general formula (I) are active for the purpose of this invention, and stable in air to allow effective application for topical use on the skin without being transformed into a different inactive substance as a result of oxidation.

Therefore, said compounds of general formula (I) have a preserving and protective action on epithelial stem cells and progenitor cells that descend from them. They can be effectively applied topically on the scalp or on the skin, depending on the desired use, in the form of suitable pharmaceutical or cosmetic compositions to obtain the above effects.

Hence, the subject of this invention is also a composition for pharmaceutical or cosmetic use for the treatment of cicatricial alopecia, containing as active ingredient at least one compound of formula (I), either as such or in the form of a pharmaceutically acceptable derivative, such as a salt, for topical administration on the scalp.

Likewise, the subject of this invention is also a composition for pharmaceutical or cosmetic use for cicatrisation of wounds containing as active ingredient at least one compound of formula (I), either as such or in the form of a pharmaceutically acceptable derivative, such as a salt, for topical administration on the skin, and also on the scalp, if the wound is located there.

Considering a different form of implementation, the subject of the invention is also a composition that includes at least one compound of formula (I) together with hair follicle or epidermal progenitor cells maintained in culture, which are, therefore, protected and preserved in said composition until the time of use. This form of implementation especially addresses the case, for instance, of preserving the integrity of said progenitor cells when they are used during trials and tests in corresponding clinical or pharmacological studies. Hence, a suitable growth medium can also be envisaged in said composition.

An additional form of implementation of the composition includes at least one compound of formula (I) together with hair follicle progenitor cells obtained from a subject in case of autologous hair transplantation, cells that are thus preserved during the period of time that precedes the autologous transplantation.

### DETAILED DESCRIPTION OF THE INVENTION

Generally, suitable forms of the composition for topical use containing, as active ingredient, at least one compound of formula (I) are, for instance, lotion, cream, serum, conditioner, mask, gel, formulated with suitable excipients for the scalp or for the skin, respectively.

If said compound of formula (I) is in the form of a pharmaceutically acceptable derivative, such as a salt, it is preferably a maleic acid salt, such as trimaleate, or a hydrochloric acid salt, such as trichlorohydrate.

Every other salt of an organic or inorganic acid that is pharmaceutically acceptable for a formulation for topical use is suitable.

A preferred compound of formula (I) for this invention is N¹-methyl-spermidine, or N-(3-amino propyl)-N¹-methyl-1,4-butandiamine (CAS Registry Number 51460-23-2), of formula:

H₂N—(CH₂)₃—N¹(CH₃)—(CH₂)₄—NH₂ (II)

used in a composition of the invention either as such or as a pharmaceutically acceptable salt such as, for instance, trimaleate (3C₄H₄O₄) or trichlorohydrate (3HCl).

Another preferred compound of formula (I) for this invention is N¹-cyclohexyl-spermidine, or N-(3-amino propyl)-N¹-cyclohexyl-1,4-butandiamine (CAS Registry Number 183070-28-2), of formula:

H₂N—(CH₂)₃—N¹(C₆H₁₁)—(CH₂)₄—NH₂ (III)

used in a composition of the invention either as such or as a pharmaceutically acceptable salt such as, for instance, trimaleate (3C₄H₄O₄) or trichlorohydrate (3HCl).

A compound of formula (I), either as such or in the form of a pharmaceutically acceptable derivative, such as a salt, is contained in a composition of the invention for topical use in a quantity that is preferably within the following weight percentage intervals, w/w (%): from 0.010 to 0.30; or weight/volume, w/v (%): from 0.0001 to 0.15.

A compound of formula (I), either as such or in the form of a pharmaceutically acceptable derivative, such as a salt, is contained in a composition of the invention to be used to preserve stem cells that are maintained in culture according to a quantity that is preferably within the following ranges: from 0.001% to 1% w/w; from 1% to 3% w/w; from 0.5 to 1.5 μM.

Some examples of compositions formulated based on the invention for topical use on the scalp or on the skin of body and face, or suitable for the preservation of stem cells maintained in culture are described below but not as a limitation. The quantities of components indicated with the INCi nomenclature are expressed in weight percentage w/w (%) or in weight per volume w/v (%), that are variable within the intervals indicated herein.

It must be said that the compound N-Methyl-N-(3-aminopropyl)tetramethylenediamine defined herein according to the INCI nomenclature corresponds to N¹-methyl-spermidine, or N-(3-amino propyl)-N¹-methyl-1,4-butandiamine of said formula (II):

### Example 1

ANDROGENETIC ALOPECIA TREATMENT
LOTION WITH SOY LECITHIN

| Component (INCI name) | Quantity w/v (%) |
|---|---|
| Lecithin (Glycine max L.) | 0.005-5.0 |
| Alcohol denat | 10.0-20.0 |
| N-Methyl-N-(3-aminopropyl) tetramethylenediamine | 0.010-0.30 |
| trimaleate | |
| Biotin | 0.01-0.10 |
| Calcium pantothenate | 0.1-3.0 |
| Rutin | 0.001-0.05 |
| PEG-40 Hydrogenated Castor Oil | 0.5-2.0 |
| Octadecyl Di-t-butyl-4-hydroxyhydrocinnamate | 0.05 |
| Parfum | 0.20 |
| Zeaxanthin | 0.002-0.01 |
| Helianthus annuus seed oil | 0.001-0.01 |
| Lactic acid | qs to pH 5.0 |
| Aqua | qs 100 mL |

### Example 2

ANDROGENETIC ALOPECIA TREATMENT LOTION

| Component (INCI name) | Quantity w/v (%) |
|---|---|
| Alcohol denat | 10.0-20.0 |
| Propylene glycol | 0.5-10% |
| Penthylene glycol | 0.5-7% |
| N-Methyl-N-(3-aminopropyl) tetramethylenediamine | 0.010-0.30 |
| trimaleate | |
| Aqua | qs 100 mL |

### Example 3

FACIAL SERUM

| Component (INCI name) | Quantity w/w (%) |
|---|---|
| Citric acid | 0.001-0.30 |
| Alcohol denat | 5.0-9.0 |
| Propylene glycol | 0.5-7.0% |
| Penthylene glycol | 0.5-7.0% |
| Sodium hydroxide | 0.001-0.30 |
| Hydroxyethylcellulose | 0.01-0.80 |
| Sorbic acid | 0.01-0.20 |
| Sorbitol | 0.10-1.00 |
| N-Methyl-N-(3-aminopropyl)tetramethylenediamine | 0.010-0.30 |
| trimaleate | |
| Limnanthes alba seed oil | 0.20-5.00 |
| Glycerin | 0.50-8.00 |
| Propanediol | 0.50-8.00 |
| Parfum | 0.10-0.70 |
| Aqua | qs 100 g |

### Example 4

MOISTURISING SERUM

| Component (INCI name) | Quantity w/w (%) |
|---|---|
| N-Methyl-N-(3-aminopropyl)tetramethylenediamine | 0.00010-0.15 |
| trimaleate | |
| Xanthan gum | 0.02-0.50 |
| Sodium alginate | 0.01-0.50 |
| Gellan gum | 0.10-0.80 |
| Citric acid | 0.001-0.30 |
| Sodium hydroxide | 0.001-0.30 |
| Glycerin | 0.50-5.00 |
| Trehalose | 0.01-0.20 |
| Sorbitol | 0.50-5.00 |
| Propanediol | 0.50-5.00 |
| Penthylene glycol | 0.50-4.00 |
| Potassium sorbate | 0.05-0.20 |
| Sodium benzoate | 0.05-0.20 |
| Parfum | 0.050-0.50 |
| Peg 40-hydrogenated castor oil | 0.10-0.80 |
| PEG-6-Caprylic/Capric Glycerides | 0.10-0.80 |
| Aqua | qs 100 g |

### Example 5

FACIAL CREAM

| Component (INCI name) | Quantity w/w (%) |
|---|---|
| Caprylic/capric triglyceride | 0.50-5.00 |
| Butyrospermum parkii butter | 0.10-0.50 |
| Oleyl erucate | 0.50-5.00 |
| Sorbitan stearate | 0.20-3.00 |
| Sucrose cocoate | 0.20-3.00 |
| Cyclopentasiloxane | 0.10-3.00 |
| Sodium hydroxymethylglycinate | 0.05-0.30 |
| Limnanthes alba seed oil | 0.20-5.00 |
| Paraffinum liquidum | 0.50-5.00 |
| Polyglyceryl-3 Rice Branate | 0.50-4.00 |
| Xanthan gum | 0.05-0.20 |
| Disodium EDTA | 0.025-0.05 |
| Xilitol | 0.50-1.50 |
| Glycerin | 0.50-3.00 |
| Sorbitol | 0.50-3.00 |
| Trehalose | 0.50-3.00 |
| Dimethicone | 0.20-6.00 |
| Cetearyl alcool | 0.20-5.00 |
| Phenoxyethanol | 0.30-0.90 |
| N-Methyl-N-(3-aminopropyl)tetramethylenediamine | 0.00010-0.15 |
| trimaleate | |
| Parfum | 0.10-0.30 |
| Lactic acid | 0.001-0.30 |
| Aqua | qs 100 g |

### Example 6

DERMATOLOGICAL GEL

| Component (INCI name) | Quantity w/w (%) |
|---|---|
| Carbomer | 0.10-2.00 |
| N-Methyl-N-(3-aminopropyl)tetramethylenediamine | 0.00010-0.15 |
| trimaleate | |
| Sodium hydroxide | 0.01-0.90 |
| Methylparaben | 0.05-0.20 |
| Propylparaben | 0.05-0.20 |
| Disodium EDTA | 0.01-0.15 |
| Hydroxypropylmethylcellulose | 0.05-1.50 |
| Propylen glycol | 2.00-15.00 |
| Lactic acid | 0.010-0.90 |
| Aqua | qs 100 g |

### Example 7

COMPOSITION FOR THE PRESERVATION OF
HAIR FOLLICLE STEM CELLS IN CULTURE

| | |
|---|---|
| CnT-07 growth medium/Dulbecco's modified eagle | 88-96.5% |
| medium 1:1 | |
| Fetal bovine serum | 2.5-10% |
| Antibiotic-antimycotic | 1% |
| N-Methyl-N-(3-aminopropyl) tetramethylenediamine | 0.001-1% |
| trimaleate | |

### Example 8

COMPOSITION FOR THE PRESERVATION OF
HAIR FOLLICLE STEM CELLS IN CULTURE

| | |
|---|---|
| CnT-07 growth medium | 86-95.5% |
| Fetal bovine serum | 2.5-10% |
| Antibiotic-antimycotic | 1% |
| N-Methyl-N-(3-aminopropyl) tetramethylenediamine | 1-3% |
| trimaleate | |

### Example 9

COMPOSITION FOR THE PRESERVATION
OF EPITHELIAL STEM CELLS

| DMEM and Ham's F12 (4:1) | 500 | ml |
|---|---|---|
| adenine | 180 | mM |
| fetal bovine serum | 10% | |
| penicillin | 100 | U/ml |
| streptomycin | 100 | mg/ml |
| hydrocortisone | 1.1 | mM |
| transferrin | 649 | nM; |
| triiodo-l-thyronine | 2 | nM |
| insulin | 862 | nM |
| EGF | 1.6 | nM |
| N-Methyl-N-(3-aminopropyl) tetramethylenediamine | 0.5-1.5 | μM |
| trihydrochloride | | |

Experimental Section

Assay on Quantitative Immunohistochemistry

The scope of the study is to determine the effects of N¹-methyl spermidine, a compound of formula (I) of this invention, on the biomarker keratin K15 (cytokeratin 15, CK15) of human epithelial hair follicle stem cells (heHFSCs). As reference on this point in the literature we can quote Kloepper JE et al. (2008), Immunophenotyping of the human bulge region: the quest to define useful in situ markers for human epithelial hair follicle stem cells and their niche. Exp Dermatol 17: 592-609.

Spermidine was used as a control.

Materials and Methods
Tissue samples

Scalp samples were collected from two women who were submitted to surgical lifting, after obtaining their informed consent. Experiments were performed in compliance with the Helsinki principles, with the Ethics Committee's approval. Details of samples are described in Table 1 below.

TABLE 1

| | Age (years) | Region of the scalp |
|---|---|---|
| Patient 1 (female) | 25 | frontal-temporal scalp |
| Patient 2 (female) | 60 | frontal-temporal scalp |

Micro-dissection of hair follicles and organ culture From said samples, follicles (HF) that are normally pigmented in anagen phase VI (the study did not include grey/white hair follicles) were microdissected from scalp skin and cultivated according to the model proposed by Philpott MP, Sanders D, Westgate G E, Kealey T (1994), Human hair growth in vitro: a model for the study of hair follicle biology. J Dermatol Sci 7: SuppIS 55-72.

A corresponding comparative test was conducted with spermidine.

N¹-methyl-spermidine of the invention or spermidine or the vehicle were changed every 48 hours with the culture medium. A summary of the study procedure is presented in Table 2 below, in which N¹-methyl spermidine, the compound of formula (II) of this invention, used in the test at concentrations of 0.5 and 1.5 μM is the active substance', while spermidine was used as a control at the concentration of 0.5 μM.

TABLE 2

| Day 0 | Day 1 | Day 2 | Day 3 | Day 4 | Day 5 | Day 6 |
|---|---|---|---|---|---|---|
| Micro- | Change of the culture | | Change of the culture | | Change of the culture | Assessments |
| dissection | medium, either alone | | medium, either alone | | medium, either alone | |
| | or with the addition of | | or with the addition of | | or with the addition of | |
| | the active substance | | the active substance | | the active substance | |

Quantitative immunohistochemistry for K15

The tyramide signal amplification method was used to assess the expression of keratin K15, as described in the abovementioned paper by Kloepper et al. (2008). The cryosections fixed in acetone were washed thrice for 5 minutes using the buffer (0.1 mol/L Tris-HCl, pH 7.5, containing 0.15 mol/L NaCl and 0.05% Tween 20) TNT (Tris-HCl NaCl Tween). Later, peroxidase was inhibited by washing with 3% H₂O₂ in phosphate buffered saline (PBS) for 15 min. Preincubation was performed with avidin and biotin for 15 minutes, and with 5% normal goat serum in TNT for 30 minutes with intermediate lavage. The Mouse Anti-Human K15 antibody (clone LHK15, Chemicon, Billerica, USA) was diluted in TNT and incubated for one night at 4° C. This incubation period was followed by a second incubation period with a secondary biotinylated Goat Anti-Mouse antibody (1:200 in TNT) for 45 minutes in RT. Streptavidin peroxidase (TSA kit, Perkin-Elmer, Boston, Mass., USA) (1:100 in TNT) was later added for 30 minutes at ambient temperature. The reaction was amplified with the FITC-tyramide amplification reagent at room temperature for 5 min (1:50 in diluent provided with the kit). Immunohistochemistry intensity was quantified with the ImageJ software (National Institutes of Health). Stain intensity of the defined reference regions of the follicle was measured and compared between the control groups and groups treated with N¹-methyl spermidine at the concentrations of 0.5 and 1.5 μM, or with spermidine 0.5 μM.
Statistical Analysis

The statistical analysis was performed using a two tailed Student t-test for samples that were not coupled. To group the two experiments, each treatment group was compared with the control group (mean value), and the relative variation in expression was calculated.

Results
Effects of N¹-methyl spermidine on the biomarker keratin K15:

the results, expressed as normalisation versus control, are reported in the two graphs of the figures of the enclosed drawings.

### DESCRIPTION OF THE DRAWINGS

FIG. 1 shows the relative intensity of immunohistochemistry for samples treated with N¹-methyl spermidine at concentrations of 0.5 and 1.5 μM, compared to the vehicle.

FIG. 2 shows the relative intensity of immunohistochemistry for samples treated with N¹-methyl spermidine at concentrations of 0.5 μM, compared to the vehicle, and in comparison with the sample treated with an equal concentration of 0.5 μM of spermidine, as reference.

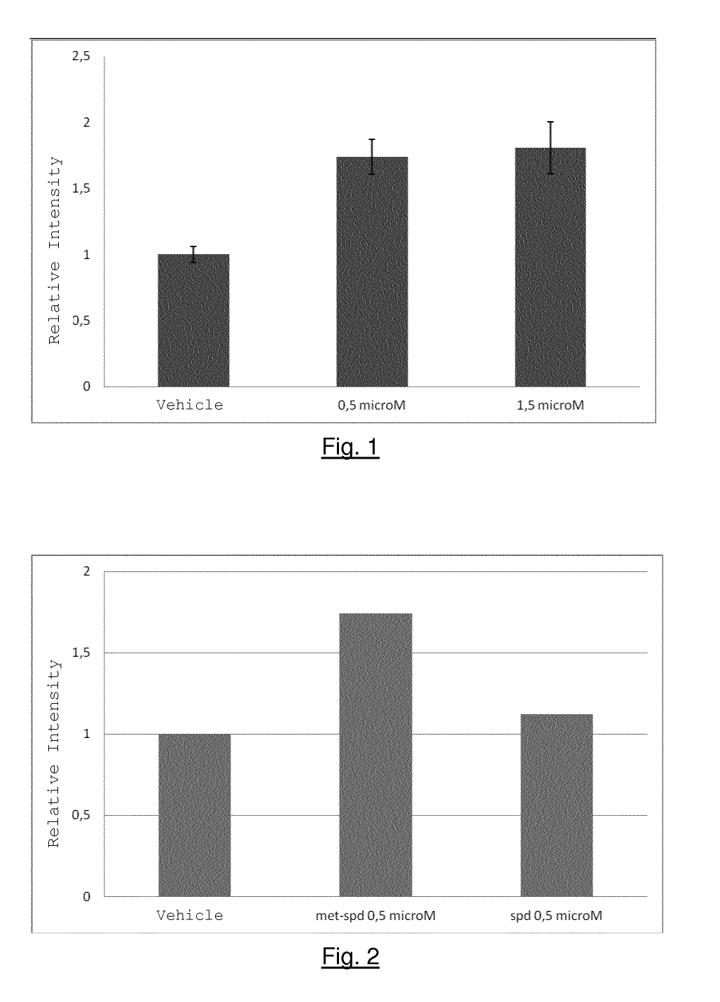

FIG. 1 shows that N¹-methyl spermidine strongly stimulates the immunoreactivity of keratin K15 at both tested concentrations, with the relative intensity being 1.739 at the concentration 0.5 μM, and 1.806 at the concentration 1.5 μM (relative intensity of the vehicle=1).

FIG. 2 shows that N¹-methyl spermidine (in the graph: met-spd) produces an inductive effect towards keratin K15 that is higher than that of an equal concentration of 0.5 μM of spermidine (in the graph: spd), the relative intensity produced by the latter being equal to 1.12.

Since N¹-methyl spermidine, a compound of formula (I) of this invention, has an evident stimulating effect on the biomarker keratin K15 (cytokeratin 15, CK15) of hair follicle stem cells, the technical effects of preservation and protection of epithelial stem cells and of progenitor cells that descend from them, consistently with the purpose of the invention, is achieved.

1-19. (canceled)

20. Method for treating humans in need of epidermal regeneration, wherein a composition comprising an effective dose of at least one of the compounds of formula (I) R-N¹-spermidine, or 1,4-butandiamin,N-(3-amino propyl)-N¹—R,
H₂N—(CH₂)₃—N¹(R)—(CH₂)₄—NH₂ (I)
wherein R is a substituent bound to the secondary amine function of spermidine, selected from:
saturated or unsaturated, linear or branched alkyl groups constituted by 1 to 6 carbon atoms, wherein one or more carbon atoms are optionally substituted by fluorine, such as methyl, ethyl, trifluoromethyl, trifluoroethyl, propyl, isopropyl, butyl, isobutyl, pentyl, hexyl, ethylene, vinyl, propylene, butylene;
aryl or arylalkyl groups, such as phenyl, naphthyl, benzyl, tolyl, wherein one or more carbon atoms are optionally substituted by fluorine, and wherein said arylalkyl groups comprise saturated or unsaturated, linear or branched alkyl groups constituted by 1 to 6 carbon atoms, wherein one or more carbon atoms are optionally substituted by fluorine, namely methyl, ethyl, trifluoromethyl, trifluoroethyl, propyl, isopropyl, butyl, isobutyl, pentyl, hexyl, ethylene, vinyl, propylene, butylene;
saturated or unsaturated cycloalkyl groups constituted by 3 to 8 carbon atoms, optionally substituted by saturated or unsaturated, linear or branched alkyl groups constituted by 1 to 6 carbon atoms, wherein one or more carbon atoms are optionally substituted by fluorine, such as methyl, ethyl, trifluoromethyl, trifluoroethyl, propyl, isopropyl, butyl, isobutyl, pentyl, hexyl, ethylene, vinyl, propylene, butylene; or a corresponding pharmaceutically acceptable salt,
is topically administered on the skin to preserve epithelial stem cells and progenitor epidermal cells.

21. Method according to claim 20 to promote epidermal cicatrisation and wound healing.

22. Method according to claim 20 for the treatment of cicatricial alopecia, wherein a composition comprising an effective dose of at least one of the compounds of formula (I) is topically administered on the scalp to preserve the hair follicle stem cells that have originated from epithelial stem cells.

23. Method according to claim 20 in which said cicatricial alopecia is one of the following: lichen planopilaris, frontal fibrosing alopecia, chronic cutaneous lupus erythematosus, keratosis follicularis, spinulosa decalvans, folliculitis decalvans.

24. Method according to claim 20, in which said compound of formula (I) is N¹-methyl-spermidine, or N-(3-amino propyl)-N¹-methyl-1,4-butandiammine of formula:
H₂N—(CH₂)₃—N¹(CH₃)—(CH₂)₄—NH₂ (II)

25. Method according to claim 20, in which said compound of formula (I) is N¹-cyclohexyl-spermidine, or N-(3-amino propyl)-N¹-cyclohexyl-1,4-butandiammine of formula:
H₂N—(CH₂)₃—N¹(C₆H₁₁)—(CH₂)₄—NH₂ (III)

26. Method according to claim 20, in which said compound of formula (I) is a trihydrochloride salt or maleic acid salt thereof.

27. Pharmaceutical or cosmetic composition to preserve and protect epithelial stem cells, and progenitor cells that derive from them, so as to promote epidermal regeneration, comprising a compound of formula (I) R—N¹-spermidine, or 1,4-butandiamin,N-(3-amino propyl)-N¹—R,
H₂N—(CH₂)₃—N¹(R)—(CH₂)₄—NH₂ (I)
wherein R is a substituent bound to the secondary amine function of spermidine, selected from:
saturated or unsaturated, linear or branched alkyl groups constituted by 1 to 6 carbon atoms, wherein one or more carbon atoms are optionally substituted by fluorine, such as methyl, ethyl, trifluoromethyl, trifluoroethyl, propyl, isopropyl, butyl, isobutyl, pentyl, hexyl, ethylene, vinyl, propylene, butylene;
aryl or arylalkyl groups, such as phenyl, naphthyl, benzyl, tolyl, wherein one or more carbon atoms are optionally substituted by fluorine, and wherein said arylalkyl groups comprise saturated or unsaturated, linear or branched alkyl groups constituted by 1 to 6 carbon atoms, wherein one or more carbon atoms are optionally substituted by fluorine, namely methyl, ethyl, trifluoromethyl, trifluoroethyl, propyl, isopropyl, butyl, isobutyl, pentyl, hexyl, ethylene, vinyl, propylene, butylene;
saturated or unsaturated cycloalkyl groups constituted by 3 to 8 carbon atoms that are optionally substituted by saturated or unsaturated, linear or branched alkyl groups formed by 1 to 6 carbon atoms, wherein one or more carbon atoms are optionally substituted by fluorine, namely methyl, ethyl, trifluoromethyl, trifluoroethyl, propyl, isopropyl, butyl, isobutyl, pentyl, hexyl, ethylene, vinyl, propylene, butylene;
or a corresponding pharmaceutically acceptable salt.

28. Composition as claimed in claim 27 for the treatment of cicatricial alopecia, wherein a compound of formula (I) is formulated with excipients that are suitable for topical application on the scalp.

29. Composition as claimed in claim 27 to promote epidermal regeneration, wherein a compound of formula (I) is formulated with excipients that are suitable for topical application on the skin.

30. Composition as claimed in claim 27 to promote cicatrisation and wound healing.

31. Composition as claimed in claim 27 comprising at least one compound of formula (I) together with hair follicle or epidermal progenitor cells in culture.

32. Composition as claimed in claim 27 for autologous transplantation of hair including at least one compound of formula (I) together with hair follicle progenitor cells.

33. Composition as claimed in claim 27, in which said compound of formula (I) is N¹-methyl-spermidine, or N-(3-amino propyl)-N¹-methyl-1,4-butandiammine of formula:
H₂N—(CH₂)₃—N¹(CH₃)—(CH₂)₄—NH₂ (II)

34. Composition as claimed in claim 27, in which said compound of formula (I) is N¹-cyclohexyl-spermidine, or N-(3-amino propyl)-N¹-cyclohexyl-1,4-butandiammine of formula:
H₂N—(CH₂)₃—N¹(C₆H₁₁)—(CH₂)₄—NH₂ (III)

35. Composition as claimed in claim 27, in which said compound of formula (I) is present in an amount by weight percentage, w/w (%), in a range from 0.0001 to 0.30.

36. Composition as claimed in claim 27, in which said compound of formula (I) is present in an amount by weight/volume percentage, w/v (%), in a range from 0.0001 to 0.15.

37. Composition as claimed in claim 27, in which said compound of formula (I) is present in an amount by weight percentage, w/w (%), in a range from 0.0001 to 0.15.
